(19) United States
(12) Patent Application Publication
(10) Pub. No.: US 2016/0184332 A1
(43) Pub. Date: Jun. 30, 2016

(54) MEDICAMENT COMPRISING A PHARMACEUTICAL COMBINATION OF DRUGS

(71) Applicant: RATIOPHARM GMBH, Ulm (DE)

(72) Inventor: Juergen RENNER, Ulm (DE)

(21) Appl. No.: 14/909,298

(22) PCT Filed: Aug. 13, 2014
(86) PCT No.: PCT/EP2014/067305

§ 371 Date: Feb. 1, 2016

Related U.S. Application Data
(60) Provisional application No. 61/865,648, filed on Aug. 14, 2013.

(30) Foreign Application Priority Data
Aug. 14, 2013 (EP) 13180343.9
Dec. 19, 2013 (EP) 13198255.5

(51) Int. Cl. A61K 31/675; A61K 31/513; A61K 31/5365

(52) U.S. Cl. CPC A61K 31/675; A61K 31/5365; A61K 31/513

(57) ABSTRACT

The present invention concerns a pharmaceutical combination comprising (a) dolutegravir, (b) emtricitabine and (c) tenofovir or a prodrug thereof, wherein preferably the compounds are present in a specific weight ratio and/or in specific amounts, for use as a medicament, preferably in treatment of viral infection/disease such as an HIV infection. Further, the present invention relates to a pharmaceutical composition comprising the pharmaceutical combination and optionally further pharmaceutical excipient(s).

The present invention concerns a pharmaceutical combination comprising (a) dolutegravir, (b) emtricitabine and (c) tenofovir or a prodrug thereof, wherein preferably the compounds are present in a specific weight ratio and/or in specific amounts, for use as a medicament, preferably in treatment of viral infection/disease such as an HIV infection. Further, the present invention relates to a pharmaceutical composition comprising the pharmaceutical combination and optionally further pharmaceutical excipient(s).

### BACKGROUND

A virus infection can be regarded as infiltration of a living organism, such as a human being, by a virus. After having been infiltrated, the organism might respond to said infiltration. For example, in humans the white blood cells of the immune system help to protect the body from viruses by destroying the cells infected by the virus. However, the destruction of the infected cells may lead to a release of intact viral particles, which can infect further so far non-infected cells and result in specific diseases or disorders of the organism.

An infiltration by the human immunodeficiency virus (HIV) may cause acquired immunodeficiency syndrome (AIDS). In that case the immune system of an organism, such as a human being, is weakened such that said system is not able to antagonize further life-threatening opportunistic infections. At present an HIV infection is said to be not curable, such that it is a main target to prevent or at least significantly slow down the proliferation of the corresponding virus. Since the treatment of an HIV infection is quite complex, combination therapies have been proposed. Combination therapies are therapies in which two or more pharmaceutical active agents are used. For example, in clinical trials a combination called “572-Trii” comprising 50 mg dolutegravir, 300 mg lamivudine and 600 mg abacavir seems to be very promising.

However, recently concerns against combination therapies have been reported. Firstly, the interaction between two or more drugs may affect the intended therapeutic activity of each drug and secondly, the interaction may increase the levels of toxic metabolites (Guidance for Industry, 1999). The interaction may also heighten or lessen the side effects of each drug. Hence, upon administration of two or more drugs to treat a disease it is unpredictable what change will occur in the negative side effects of each drug. Additionally, it is difficult to accurately predict when the effects of the interaction between the two or more drugs will become manifest. For example, metabolic interactions between drugs may become apparent upon initial administration of the second drug, after the two have reached a steady state concentration or upon discontinuation of one of the drugs (Guidance for Industry, 1999).

Raffi Francois et al, Lancet (North American edition), vol. 381, no. 9868, March 2013, pages 735-743, describes a study wherein the HIV integrase inhibitors, dolutegravir and raltegravir are compared. In particular either 50 mg of dolutegravir or 400 mg of raltegravir in combination with two known NRTI-backbones (coformulated tenofovir/emtricitabine or abacavir/lamivudine), respectively, are administered to patients with HIV-1 infection. As a result of the study it turned out that dolutegravir can be regarded as suitable alternative to raltegravir.

However, the combinations suggested in Raffi still seem to be improvable in view of their efficiency and/or safety. In particular, the reduction of possible side effects, e.g. when administered to older people or to people having an impaired immune system, is desirable.

Thus, it was an object of the present invention to overcome the above-illustrated drawbacks. In particular, it was an object of the invention to provide an improved medicament or treatment regime for the treatment of viral diseases, in particular HIV. Preferably, it was an object of the present invention to provide a medicament which leads to advantageous therapeutic effects when treating viral diseases, in particular HIV. Further, undesirable side effects should be prevented or at least significantly reduced.

Further, it was an object to provide a medicament wherein the total amount of pharmaceutical active agent(s) is suitable for preparing a dosage form having a comfortable size such that patient compliance can be improved.

The objects of the present invention have been unexpectedly solved by a combination comprising (a) dolutegravir, (b) emtricitabine and (c) tenofovir, having a specific weight ratio of (a) dolutegravir to emtricitabine (b) and preferably being administered in specific amounts.

### SUMMARY OF THE INVENTION

This invention provides a pharmaceutical combination comprising (a) dolutegravir, (b) emtricitabine and (c) tenofovir for use as a medicament.

This invention also provides a pharmaceutical composition comprising (a) dolutegravir, (b) emtricitabine and (c) tenofovir, wherein the weight ratio of (a) dolutegravir and (b) emtricitabine is from 1:1 to 1:3.

This invention in addition provides a method of treating a subject afflicted with a viral infection, preferably afflicted with an HIV infection, comprising periodically administering to the subject an amount of (a) dolutegravir, (b) emtricitabine and (c) tenofovir, wherein the amounts when taken together are effective to treat the subject, wherein preferably the subject is a human.

### DETAILED DESCRIPTION OF THE INVENTION

The pharmaceutical combination of the present invention comprises (a) dolutegravir as a first active pharmaceutical ingredient.

The chemical name of (a) dolutegravir, also known as S/GSK1349572 or just “572”, is (4R,12aS)—N-(2,4-difluorobenzyl)-7-hydroxy-4-methyl-6,8-dioxo-3,4,6,8,12,12a-hexa-hydro-2H-pyrido[1′,2′:4,5]pyrazino[2,1-b][1,3]oxazine-9-carboxamide. Dolutegravir is reported to have anti-viral activity, since it is considered to be an integrase inhibitor. Integrase is a viral enzyme for integrating (retro)viral DNA into the genome of the host cell and the inhibition of the integrase has a massive influence on the replication of the retrovirus. By inhibiting said integrase the replication of the retrovirus can be significantly reduced or even prevented. Thus, dolutegravir is assumed to be useful for the treatment of infections/diseases caused by viruses such as hepatitis B and HIV. In particular dolutegravir is assumed to be useful for the treatment of HIV infections and/or AIDS. 4R,12aS)—N-(2,4-difluorobenzyl)-7-hydroxy-4-methyl-6,8-dioxo-3,4,6,8,12,12a-hexa-hydro-2H-pyrido[1′,2′:4,5]pyrazino[2,1-b][1,3]oxazine-9-carboxamide (dolutegravir) is represented by the following chemical structure:

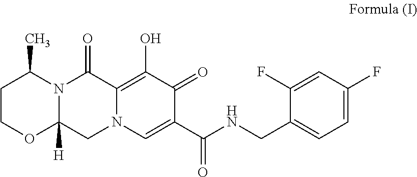 [SMILES: C[C@@H]1CCO[C@H]2Cn3cc(C(=O)NCc4ccc(F)cc4F)c(=O)c(O)c3C(=O)N12]

In this regard it is noted that in the present invention the term dolutegravir comprises 4R,12aS)—N-(2,4-difluorobenzyl)-7-hydroxy-4-methyl-6,8-dioxo-3,4,6,8,12,12a-hexahydro-2H-pyrido[1′,2′:4,5]pyrazino[2,1-b][1,3]oxazine-9-carboxamide according to Formula (I). In addition, the term “dolutegravir” comprises all the pharmaceutically acceptable salts, hydrates and/or solvates thereof. Dolutegravir can be obtained according to the procedures as for example outlined in WO 2006/116764 or WO 2010/068253.

Acids, which are used to prepare pharmaceutically acceptable acid addition salts, are preferably those which form non-toxic acid addition salts. Examples of the acid addition salts are mineral acid salts such as hydrochloride, hydrobromide, hydroiodide, hemisulfate, sulfate, nitrate, dihydrogenphosphate, hydrogen phosphate, hydrogen carbonate, carbonate or perchlorate salts; organic acid salts such as formate, acetate, propionate, lactates, maleates, fumarates, tartrates, malates, citrates, ascorbates; acidic amino acid salts such as aspartates or glutamates and methanesulfonates, benzenesulfonates, or p-toluenesulfonates.

Examples of suitable basic salts include alkali metal salts such as sodium or potassium salts; earth-alkali metal salts such as calcium or magnesium salts; ammonium salts; aliphatic amine salts such as trimethylamine, triethylamine, dicyclohexylamine, ethanolamine, diethanolamine, triethanolamine or procaine salts; aralkyl amine salts such as N,N-dibenzylethylonediamine salts; heterocyclic aromatic amine salts such as pyridine salts, methyl pyridine salts, quinoline salts or isoquinoline salts; quaternary ammonium salts such as tetramethylammonium salts, tetraethylammonium salts, benzyltrimethylammonium salts, benzyltriethyl-ammonium salts, benzyltributylammonium salts, methyltrioctylammonium salts or tetrabutylammonium salts; and basic amino acid salts such as arginine salts or lysine salts.

Preferably, dolutegravir is in the form of a pharmaceutically acceptable salt. More preferred, dolutegravir is in the form of an alkali metal salt such as sodium or potassium salt or in the form of an earth-alkali metal salt such as calcium or magnesium salt. More preferred, dolutegravir is in the form of a sodium salt or a magnesium salt. Particularly preferred, dolutegravir is used in form of the sodium salt, especially the mono-sodium salt.

It is alternatively particularly preferred that dolutegravir is used in its free form (not as salt).

The sodium salt of dolutegravir and a specific crystalline form of this sodium salt or a hydrate thereof are disclosed in WO 2010/068253. Amorphous dolutegravir sodium is described in WO 2013/038407.

Further, it is preferred that dolutegravir is present in a non-solvated/non-hydrated form.

In a preferred embodiment of the present invention all numbers referring to the weight (mg) or weight-% of dolutegravir are based on the weight of dolutegravir in its free form.

In the combination of the present invention, dolutegravir as the active ingredient can be provided in amorphous form, in crystalline form or as a mixture of both forms. Preferably, dolutegravir is present in crystalline form.

It is reported that its primary route of metabolism is glucuronidation. Dolutegravir is considered to be a substrate of CYP3A4, but only to a minor extent of about 15%. Further, dolutegravir demonstrates induction or inhibition of cytochrome P450 (CYP) isozymes in vitro.

The pharmaceutical combination of the present invention comprises (b) emtricitabine as a second active pharmaceutical ingredient.

The chemical name of (b) emtricitabine is 5-fluoro-1-(2R,5S)-[2-(hydroxymethyl)-1,3 oxathiolan-5-yl]cytosine. Emtricitabine is reported to be a substance with anti-viral activity since it is considered to be a nucleoside reverse transcriptase inhibitor. Reverse transcriptase is a viral DNA polymerase that is required for replication of the virus. The reverse transcriptase allows copying the single stranded RNA genome into a double stranded viral DNA, which is subsequently incorporated into the DNA of the host cell. Having been integrated in the host cell genome, viral particles are produced which in turn can infect so far uninfected host cells.

Nucleoside analog reverse-transcriptase inhibitors (also known as NARTIs or NRTIs) are considered to inhibit/prevent the activity of reverse transcriptase by blocking its enzymatic function.

By inhibiting said reverse transcriptase the replication of the retrovirus can be significantly reduced or even prevented. Thus, emtricitabine is assumed to be useful for the treatment of infections/diseases caused by viruses such as hepatitis B and HIV. In particular emtricitabine is assumed to be useful for the treatment of HIV infections and/or AIDS. Emtricitabine is also known as 5-fluoro-1-(2R,5S)-[2-(hydroxymethyl)-1,3 oxathiolan-5-yl]cytosine and is represented by the following chemical structure:

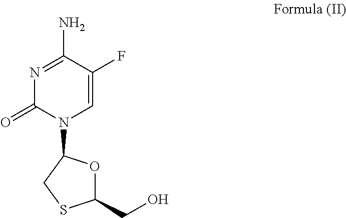 [SMILES: Nc1nc(=O)n([C@@H]2CS[C@H](CO)O2)cc1F]

In this regard it is noted that in the present invention the term “emtricitabine” comprises 5-fluoro-1-(2R,5S)-[2-(hydroxymethyl)-1,3 oxathiolan-5-yl]cytosine according to Formula (II). In addition, the term “emtricitabine” comprises all the pharmaceutically acceptable salts, hydrates and/or solvates thereof. Emtricitabine can for example be obtained according to the procedures as outlined in EP 0 513 200 B2.

Emtricitabine possesses two chiral centers. Preferably emtricitabine is in the form of the cis-(−)-enantiomer.

Acids which are used to prepare pharmaceutically acceptable acid addition salts are preferably those which form non-toxic acid addition salts. Examples of the acid addition salts are mineral acid salts such as hydrochloride, hydrobromide, hydroiodide, hemisulfate, sulfate, nitrate, dihydrogen phosphate, hydrogen phosphate, hydrogen carbonate, carbonate or perchlorate salts; organic acid salts such as formate, acetate, propionate, lactates, maleates, fumarates, tartrates, malates, citrates, ascorbates; acidic amino acid salts such as aspartates or glutamates and methanesulfonates, benzenesulfonates, or p-toluenesulfonates.

In a preferred embodiment emtricitabine is present in the form of its free base, i.e. not in the form of an acid addition salt.

Further, it is preferred that emtricitabine is present in a non-solvated/non-hydrated form.

In a preferred embodiment of the present invention all numbers referring to the weight (mg) or weight-% of emtricitabine are based on the weight of emtricitabine in its free form.

In the combination of the present invention, emtricitabine as the active ingredient can be provided in amorphous form, in crystalline form or as a mixture of both forms. Preferably, emtricitabine is present in crystalline form. Different polymorphic forms of crystalline emtricitabine are known. Preferably crystalline emtricitabine is polymorph form I.

It is reported that there is limited metabolism. The biotransformation of emtricitabine includes oxidation of the thioether-containing moiety to form sulphoxidediatereomers and conjugation with glucuronic acid to form 2′-O-glucuronide. Emtricitabine does not inhibit in vitro drug metabolism mediated by the following human CYP 450 isoenzymes: 1A2, 2A6, 2B6, 2C9, 2C19, 2D6 and 3A4. Further, emtricitabine does not inhibit uridine-5-5′-diphosphoglucuronyl transferase, the enzyme responsible for glucuronidation.

The pharmaceutical combination of the present invention comprises (c) tenofovir as a third active pharmaceutical ingredient.

The chemical name of (c) tenofovir is (R)-(1-(6-amino-9H-purin-9-yl)propan-2yloxy)methyl phosphonic acid. Like the above compounds (a) dolutegravir and (b) emtricitabine, compound (c) tenofovir is reported to be a substance with anti-viral activity. Tenofovir is considered to be a nucleotide reverse transcriptase inhibitor (NtRTI). NtRTIs, such as tenofovir, allegedly have a mode of action being quite similar to the ones of the nucleoside reverse transcriptase inhibitors (NRTI), such as emtricitabine mentioned above. Substantially, NtRTIs should disrupt the construction of a new piece of proviral DNA and thus reduce/prevent the replication of the (retro)virus. Consequently, tenofovir is assumed to be useful for the treatment of infections/diseases caused by viruses such as hepatitis B, HIV and/or AIDS, in particular HIV and/or AIDS. (R)-(1-(6-amino-9H-purin-9-yl)propan-2yloxy)methyl phosphonic acid (tenofovir) is represented by the following chemical structure:

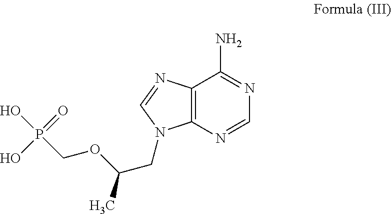 [SMILES: C[C@H](Cn1cnc2c(N)ncnc21)OCP(=O)(O)O]

In this regard it is noted that in the present invention the term “tenofovir” comprises (R)-(1-(6-amino-9H-purin-9-yl)propan-2yloxy)methyl phosphonic acid according to Formula (III). In addition the term “tenofovir” comprises all the pharmaceutically acceptable salts, hydrates and/or solvates thereof. Tenofovir is inter alia described in WO 94/03467.

Acids which are used to prepare pharmaceutically acceptable acid addition salts are preferably those which form non-toxic acid addition salts. Examples of the acid addition salts are mineral acid salts such as hydrochloride, hydrobromide, hydroiodide, hemisulfate, sulfate, nitrate, dihydrogen phosphate, hydrogen phosphate, hydrogen carbonate, carbonate or perchlorate salts; organic acid salts such as formate, acetate, propionate, lactates, maleates, fumarates, tartrates, malates, citrates, ascorbates; acidic amino acid salts such as aspartates or glutamates and methanesulfonates, benzenesulfonates, or p-toluenesulfonates.

Examples of suitable basic salts include alkali metal salts such as sodium or potassium salts; earth-alkali metal salts such as calcium or magnesium salts; ammonium salts; aliphatic amine salts such as trimethylamine, triethylamine, dicyclohexylamine, ethanolamine, diethanolamine, triethanolamine or procaine salts; aralkyl amine salts such as N,N-dibenzylethylonediamine salts; heterocyclic aromatic amine salts such as pyridine salts, methyl pyridine salts, quinoline salts or isoquinoline salts; quaternary ammonium salts such as tetramethylammonium salts, tetraethylammonium salts, benzyltrimethylammonium salts, benzyltriethyl-ammonium salts, benzyltributylammonium salts, methyltrioctylammonium salts or tetrabutylammonium salts, and basic amino acid salts such as arginine salts or lysine salts.

Preferably, tenofovir is in not in the form of a pharmaceutically acceptable salt, i.e. neither in the form of an acid addition salt nor in the form of a basic addition salt.

Further, it is preferred that tenofovir is present in a non-solvated/non-hydrated form.

In a preferred embodiment of the present invention all numbers referring to the weight (mg) or weight-% of tenofovir are based on the weight of tenofovir in its free form.

In the combination for use as a medicament of the present invention, tenofovir as the active ingredient can be provided in amorphous form, in crystalline form or as a mixture of both forms. Preferably, tenofovir is present in crystalline form.

In a preferred embodiment tenofovir is used in form of a prodrug of tenofovir, preferably as tenofovir disoproxil, or pharmaceutically acceptable salts thereof like e.g. tenofovir disoproxil fumarate, tenofovir alafenamide and/or hexadecyloxypropyl tenofovir. Generally, a prodrug can be regarded as a substance that is administered to a subject (preferably a human) in a pharmacologically inactive or pharmacologically less than fully active form, and is subsequently converted in the body of the subject to an active drug, preferably through metabolic processes occurring in the body of the subject. In other words, a prodrug usually serves as a type of ‘precursor’ to the intended drug.

In a particularly preferred embodiment the prodrug of tenofovir is tenofovir disoproxil. The chemical name of tenofovir disoproxil is (R)-(((1-(6-amino-9H-purin-9-yl)propane-2-yloxy)methyl)phosphoryl)bis(oxy)bis(methylene) isopropyl dicarbonate and is represented by the following chemical structure:

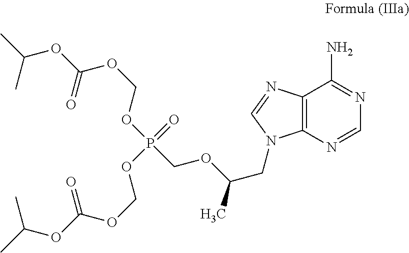 [SMILES: CC(C)OC(=O)OCOP(=O)(CO[C@H](C)Cn1cnc2c(N)ncnc21)OCOC(=O)OC(C)C]

In this regard it is noted that in the present invention the term “tenofovir disoproxil” comprises (R)-(((1-(6-amino-9H-purin-9-yl)propane-2-yloxy)methyl)phosphoryl)-bis(oxy)bis(methylene) isopropyl dicarbonate according to Formula (Ma). In addition, the term “tenofovir disoproxil” comprises all the pharmaceutically acceptable salts, hydrates and/or solvates thereof. Tenofovir disoproxil and its preparation is inter alia described in EP 0 915 894 B1.

Acids which are used to prepare pharmaceutically acceptable acid addition salts are preferably those which form non-toxic acid addition salts. In a particularly preferred embodiment tenofovir disoproxil is present in form of its hemifumarate or fumarate, especially its fumarate. Tenofovir disoproxil fumarate is also known as 9-[(R)-2-[[bis[[(isopropoxycarbonyl)oxy]methoxy]-phosphinyl]methoxy]propyl]adenine fumarate (1:1) (IUPAC) and is represented by the following chemical structure:

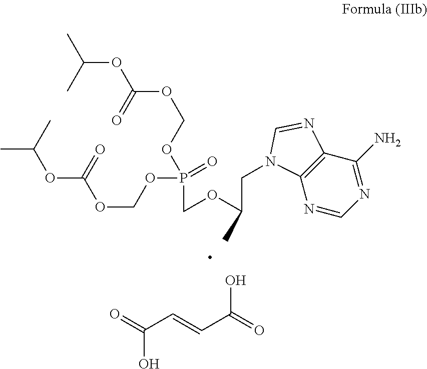

In a preferred embodiment of the present invention all numbers referring to the weight (mg) or weight-% of tenofovir disoproxil are based on the weight of tenofovir disoproxil fumarate.

In an alternative particularly preferred embodiment the prodrug of tenofovir is tenofovir alafenamide, also known as GS 7340. The chemical name of tenofovir alafenamide is (R)-isopropyl 2-((S)—(((R)-1-(6-amino-9H-purin-9-yl)propan-2-yloxy)methyl)(phenoxy)phosphorylamino)propanoate. Tenofovir alafenamide is also known as L-Alanine, N—[(S)-[[(1R)-2-(6-amino-9H-purin-9-yl)-1-methylethoxy]methyl]phenoxyphosphinyl]-, 1-methylethyl ester and is represented by the following chemical structure:

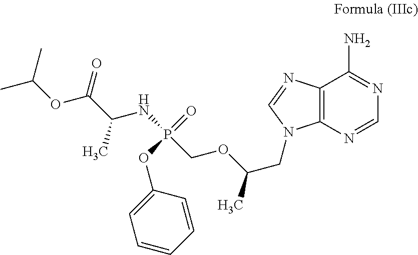 [SMILES: CC(C)OC(=O)[C@@H](C)N[P@](=O)(CO[C@H](C)Cn1cnc2c(N)ncnc21)Oc1ccccc1]

In this regard it is noted that in the present invention the term “tenofovir alafenamide” comprises (R)-isopropyl 2-((S)—(((R)-1-(6-amino-9H-purin-9-yl)propane-2-yloxy)methyl)(phenoxy)phosphorylamino)propanoate according to Formula (Mc). In addition, the term “tenofovir alafenamide” comprises all the pharmaceutically acceptable salts, hydrates and/or solvates thereof. Tenofovir alafenamide and its preparation are inter alia described in WO 2013/025788 A1.

Acids which are used to prepare pharmaceutically acceptable acid addition salts are preferably those which form non-toxic acid addition salts. In a particularly preferred embodiment tenofovir alafenamide is present in form of its hemifumarate or fumarate, especially its hemifumarate.

In a preferred embodiment of the present invention all numbers referring to the weight (mg) or weight-% of tenofovir alafenamide are based on the weight of tenofovir alafenamide as free base.

In an alternative particularly preferred embodiment the prodrug of tenofovir is hexadecyloxypropyl tenofovir, also known as CMX157. The chemical name of hexadecyloxypropyl tenofovir is 3-(hexadecyloxy)propyl hydrogen ((R)-1-(6-amino-9H-purin-9-yl)propan-2-yloxy)methyl)phosphonate. Hexadecyloxypropyl tenofovir is also known as hexadecyloxypropyl-9-(R)-[2-(phosphonomethoxy)propyl]adenine and is represented by the following chemical structure:

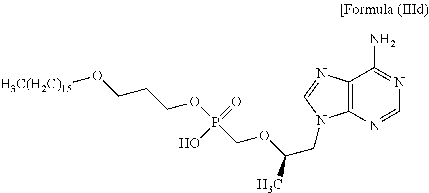

In this regard it is noted that in the present invention the term “hexadecyloxypropyl tenofovir” comprises (3-(hexadecyloxy)propyl hydrogen ((R)-1-(6-amino-9H-purin-9-yl)propan-2-yloxy)methyl)phosphonate according to Formula (IIId). In addition, the term “hexadecyloxypropyl tenofovir” comprises all the pharmaceutically acceptable salts, hydrates and/or solvates thereof. “Hexadecyloxypropyl tenofovir” is inter alia described in WO 2011/01171 A1.

In a preferred embodiment of the present invention all numbers referring to the weight (mg) or weight-% of hexadecyloxypropyl tenofovir are based on the weight of hexadecyloxypropyl tenofovir as free base.

The combination of the present invention preferably does not show any significant pharmacokinetic interaction of dolutegravir, emtricitabine and tenofovir.

In a particularly preferred embodiment the pharmaceutical combination of the present invention comprises dolutegravir, emtricitabine and tenofovir as the sole pharmaceutically active agents.

In a particularly preferred embodiment the pharmaceutical combination of the present invention comprises dolutegravir, emtricitabine and tenofovir in combination with further pharmaceutically active agent(s).

In other words, the combination of the present invention preferably can be referred to as a pharmaceutical composition comprising (a) dolutegravir, (b) emtricitabine and (c) tenofovir, wherein (a) dolutegravir and (b) emtricitabine are present in a specific weight ratio, for use as a medicament. A further subject of the present invention is a method for treating a viral infection, comprising administering to a subject in need thereof the pharmaceutical combination or composition of the present invention, wherein the viral infection preferably comprises an HIV-infection.

In the pharmaceutical combination of the present invention the weight ratio of (a) dolutegravir to (b) emtricitabine is from 1:1 to 1:3, preferably from 1:1.2 to 1:2.8, more preferably 1:1.5 to 1:2.5.

In a particularly preferred embodiment the weight ratio of (a) dolutegravir to (b) emtricitabine can be 1:2.

In the pharmaceutical combination of the present invention the weight ratio of (a) dolutegravir to (c) tenofovir may depend on the type of tenofovir prodrug and/or salt.

In case dolutegravir (in free form) and tenofovir disoproxil is used, the weight ratio of dolutegravir to (c) tenofovir disoproxil usually might be from 1:1.9 to 1:4.5, preferably 1:2.2 to 1:4.2, more preferably 1:2.4 to 1:4.

In the preferred embodiment where tenofovir disoproxil fumarate is used, the weight ratio of dolutegravir to (c) tenofovir disoproxil fumarate usually might be from 1:2 to 1:5, preferably 1:2.25 to 1:4.5, more preferably 1:2.5 to 1:4.

In a particularly preferred embodiment the weight ratio of (a) dolutegravir to (c) tenofovir disoproxil fumarate can be 1:3.

In an alternatively preferred embodiment the pharmaceutical combination of the present invention comprises (a) dolutegravir, (b) emtricitabine and (c) tenofovir alafenamide in which the weight ratio of (a) dolutegravir to (b) emtricitabine to (c) tenofovir alefanamide can be from 1:1:0.15 to 1:8:1, preferably from 1:2:0.16 to 1:8:0.8, and more preferably from 1:2:0.17 to 1:6:0.75, especially 1:4:0.19 to 1:4:0.60.

In a preferred embodiment of the pharmaceutical combination of the present invention the amount of dolutegravir administered is 20 to 75 mg per day, preferably 25 to 70 mg per day, more preferably 35 to 65 mg per day, in particular 40 to 60 mg per day. The amounts are based on dolutegravir in free form (not in form of a salt or a prodrug).

It is further preferred that the amount of emtricitabine administered is 50 to 150 mg per day, preferably 60 to 140 mg per day, more preferably 65 to 130 mg per day, in particular 75 to 125 mg per day. The amounts are based on emtricitabine in free form (not in form of a salt or a prodrug).

It is further preferred that the amount of tenofovir administered preferably is 5 to 300 mg per day, more preferably 7.5 to 250 mg per day, even more preferably 30 to 200 mg per day, in particular 40 to 175 mg per day. The amounts are based on tenofovir in free form (not in form of a salt or a prodrug).

In a preferred embodiment tenofovir is used in the form of tenofovir disoproxil fumarate. It is further preferred that the amount of tenofovir disoproxil fumarate administered preferably is 100 to 250 mg per day, more preferably 110 to 225 mg per day, even more preferably 120 to 200 mg per day, in particular 125 to 175 mg per day.

In case that the tenofovir is administered as tenofovir alafenamide, the amount of tenofovir alafenamide administered preferably is 0.5 to 50 mg per day, more preferably 1 to 45 mg per day, even more preferably 3 to 44 mg per day, in particular 5 to 42 mg per day.

As can be seen from above, the total amount of dolutegravir, emtricitabine and tenofovir disoproxil fumarate comprised in the present pharmaceutical combination for use as a medicament is between 170 and 475 mg per day, in particular 240 to 360 mg per day.

As can be seen from the above, the total amount of dolutegravir, emtricitabine and tenofovir alafenamide comprised in the present pharmaceutical combination for use as a medicament is between 70.5 and 425 mg per day, in particular 192 to 326 mg per day.

In one embodiment, the pharmaceutical combination for use as a medicament is administered via oral administration. In alternative embodiments it is preferred that the pharmaceutical combination for use as a medicament is administered per injection or infusion, e.g. intravenous, subcutaneously or intramuscular. In such a case the APIs can for example be embedded in a (degradable) biopolymer such as a polysaccharides, lipids and fibres.

In another particularly preferred embodiment, the combination of the present invention is administered once daily. Alternatively, a twice or three times daily administration can be applied.

Generally, the combination of the present invention is suitable for treating any human being having an HIV infection. In a further preferred embodiment the combination of the present invention is used for treating a HIV infection in human patients of the HLA-B 5701 genotype. HLA-B (major histocompatibility complex, class I, B) is a human gene that provides instructions for producing a protein that plays a critical role in the immune system. Among humans with HIV infection, a version of HLA-B designated HLA-B*5701 can be treated beneficially.

Further, in a preferred embodiment the combination of the present invention is used for treating HIV in patients suffering from swallowing disorders. Swallowing disorders (dysphagia) can result from congenital abnormalities, structural damage, and/or medical conditions. Swallowing problems are a common complaint among older individuals, and the incidence of dysphagia is higher in the elderly, in patients who have had strokes, and in patients who are admitted to acute care hospitals or chronic care facilities. Thus, in a preferred embodiment the combination of the present invention is used for treating HIV in patients being older than 50 years, in particular older than 60 years, e.g. 60 to 90 years. In another preferred embodiment the present invention is used to treat patients younger than 15 years, in particular younger than 14 years, e.g. 5 to 13 years.

A further subject of the present invention is a pharmaceutical composition comprising the combination of the present invention, i.e. a pharmaceutical composition comprising (a) dolutegravir, (b) emtricitabine and (c) tenofovir and optionally pharmaceutical excipients.

In a preferred embodiment the pharmaceutical composition comprises
(i) 20 to 75 mg, preferably 25 to 70 mg, more preferably 35 to 65 mg, in particular 40 to 60 mg dolutegravir (a);
(ii) 50 to 150 mg, preferably 60 to 140 mg, more preferably 65 to 130 mg, in particular 75 to 125 mg emtricitabine (b);
(iii) 100 to 250 mg, more preferably 110 to 225 mg, even more preferably 120 to 200 mg, in particular 125 to 175 mg tenofovir (c), in particular tenofovir disoproxil fumarate; and
(iv) pharmaceutical excipients.

In a preferred embodiment the pharmaceutical composition comprises
(i) 20 to 75 mg, preferably 25 to 70 mg, more preferably 35 to 65 mg, in particular 40 to 60 mg dolutegravir (a);
(ii) 50 to 300 mg, preferably 100 to 280 mg, more preferably 125 to 260 mg, in particular 150 to 250 mg emtricitabine (b);
(iii) 0.5 to 50 mg, more preferably 1 to 45 mg, even more preferably 3 to 44 mg, in particular 5 to 42 mg tenofovir (c), in particular tenofovir alafenamide; and
(iv) pharmaceutical excipients.

In a preferred embodiment the pharmaceutical composition of the present invention comprises

50 mg dolutegravir, 50 mg emtricitabine and 150 mg tenofovir (c), in particular tenofovir disoproxil fumarate, or
50 mg dolutegravir, 100 mg emtricitabine and 150 mg tenofovir (c), in particular tenofovir disoproxil fumarate, or
50 mg dolutegravir, 150 mg emtricitabine and 150 mg tenofovir (c), in particular tenofovir disoproxil fumarate, or
25 mg dolutegravir, 100 mg emtricitabine and 250 mg tenofovir (c), in particular tenofovir disoproxil fumarate, or
25 mg dolutegravir, 100 mg emtricitabine and 200 mg tenofovir (c), in particular tenofovir disoproxil fumarate, or
25 mg dolutegravir, 100 mg emtricitabine and 150 mg tenofovir (c), in particular, tenofovir disoproxil fumarate.

In an alternatively preferred embodiment the pharmaceutical composition of the present invention comprises

about 50 mg dolutegravir, about 200 mg emtricitabine and about 25 mg tenofovir (c), in particular tenofovir alafenamide, or
about 25 mg dolutegravir, about 200 mg emtricitabine and about 25 mg tenofovir (c), in particular tenofovir alafenamide, or
about 50 mg dolutegravir, about 100 mg emtricitabine and about 25 mg tenofovir (c), in particular tenofovir alafenamide, or
about 50 mg dolutegravir, about 200 mg emtricitabine and about 10 mg tenofovir (c), in particular tenofovir alafenamide, or
about 50 mg dolutegravir, about 100 mg emtricitabine and about 10 mg tenofovir (c), in particular tenofovir alafenamide,
wherein the amount of tenofovir alafenamide is based of tenofovir alafenamide free base or tenofovir alafenamide hemifumarate.

In a preferred embodiment the term “about” indicates that the corresponding value can vary plus/minus 10%, in particular plus/minus 5%.

For the foregoing embodiments, each embodiment disclosed herein is contemplated as being applicable to each of the other disclosed embodiments. For example, the explanation given above for preferred embodiments of the combination or for each of the drugs of the combination also applies to the composition of the present invention.

In a preferred embodiment the pharmaceutical composition can preferably comprise one or more pharmaceutical excipient(s). The pharmaceutical excipients are excipients with which the person skilled in the art is familiar, such as those which are described in the European Pharmacopoeia (Ph.Eur.) and/or in the US Pharmacopoeia (USP).

Examples of suitable pharmaceutical excipients are glidants (d), fillers (e), disintegrants (f), binders (g), lubricants (h) and surfactants (j).

Glidants (d) can be used to improve the flowability. For example, talc can be used as glidant. More preferably, colloidal silicon dioxide (for example Aerosil) is used. Preferably, the glidant can be present in an amount of up to 3 wt %, preferably in amount of 0.05 to 2.5 wt %, more preferably 0.1 to 2.0 wt %, in particular 0.2 to 1.5 wt % based on the total weight of the oral dosage form. Preferably, the colloidal silicon dioxide has a specific surface area of 50 to 400 m²/g, measured by gas adsorption according to Ph. Eur., 6.0, Chapter 2.9.26.

Fillers (e) can be used to increase the bulk volume and weight to a limit at which a dosage form can be formed. Fillers may fulfil several requirements, such as being chemically inert, non-hygroscopic, biocompatible, easily processable and may possess good biopharmaceutical properties. Preferred fillers are for example lactose, sucrose, glucose, mannitol, maltodextrin, dextrin, dextrose, hydrogenated vegetable oil and/or cellulose derivatives, such as microcrystalline cellulose and silicified microcrystalline cellulose, and mixtures thereof. More preferred are lactose, mannitol, microcrystalline cellulose and silicified microcrystalline cellulose, particularly lactose, microcrystalline cellulose and silicified microcrystalline cellulose, especially microcrystalline cellulose.

The fillers can be present in the oral dosage form of the present invention in an amount of 5 to 35 wt %, preferably 8 to 30 wt %, more preferably 12 to 20 wt % and still more preferably 10 to 15 wt % of the total weight of the dosage form.

Disintegrants (f) are reported to be compounds which can enhance the ability of the intermediate to break into smaller fragments when in contact with a liquid preferably water. Preferred disintegrants are sodium carboxymethyl starch (croscarmellose sodium) cross-linked polyvinylpyrrolidone, sodium carboxymethyl glycolate, swelling polysaccharides, for example soy polysaccharide, carrageenan, agar, pectin, starch and derivatives thereof, proteins, for example formaldehyde-casein, sodium bicarbonate or mixtures thereof.

Disintegrants can be present in an amount of 0 to 15 wt %, preferably 1 to 12 wt %, more preferably 2 to 10 wt % and still more preferably 3 to 6 wt %, based on the total weight of the dosage form.

Binders (g) or adhesives are reported to be substances that ensure that granulates or tablets can be formed with the required mechanical strength. Binders can be for example starch, sucrose, gelatine, polyvinylpyrrolidone, cellulose derivatives, such as hydroxypropyl methylcellulose, or mixtures thereof. Binders can preferably be present in amounts of 1 to 10 wt %, preferably of 2 to 5 wt %, in particular of 2.5 to 4.5 wt % based on the total weight of the dosage form.

Lubricants (h) are generally used in order to reduce sliding friction. In particular, the intention is to reduce the sliding friction found during tablet pressing between the punch moving up and down in the die and the die wall on the one hand and between the edge of the tablet and the die wall on the other hand. Suitable lubricants are for example stearic acid, adipic acid, sodium stearyl fumarate and/or magnesium stearate. Preferably, lubricants can be present in an amount of 0 to up to 3 wt %, more preferably of 0.1 to 2.5 wt %, in particular of 0.2 to 2 wt % based on the total weight of the dosage form.

Surfactants (j) can preferably be substances which lower the surface tension or the interfacial tension between two phases, thus enabling or supporting the formation of dispersions or working as a solubilizer. Surfactants can preferably be present in amounts of 0.3 to 5 wt %, preferably of 0.5 to 4 wt %, in particular of 1 to 3 wt % based on the total weight of the dosage form.

In this regard it is generally noted that due to the nature of pharmaceutical excipients it cannot be excluded that a certain compound meets the requirements of more than one of the components (d) to (j).

In a preferred embodiment the pharmaceutical composition of the present invention can be considered as a unit dosage form comprising

(i) 20 to 75 mg dolutegravir,
(ii) 50 to 150 mg emtricitabine,
(iii) 0.5 to 400 mg tenofovir or a prodrug thereof and optionally
(iv) pharmaceutical excipient(s).

The pharmaceutical composition of the present invention can be preferably a solid oral dosage form. It is further preferred that the dosage form of the invention is a capsule or a tablet, in particular a tablet.

A further subject of the present invention is a kit for the treatment of a viral infection, in particular an HIV-infection, comprising

(i) 20 to 75 mg dolutegravir,
(ii) 50 to 150 mg emtricitabine,
(iii) 0.5 to 400 mg tenofovir or a prodrug thereof.

This invention is illustrated by the following examples.

### Examples

### Antiviral Activity

The antiviral activity of the different combinations of pharmaceutically active agents was determined using cell from CEM-M7 cell line. The CEM-M7 cell line was a suspension T-cell line and expressed CD4, CXCR4 and CCR5 and was stably transduced with a long terminal repeat (LTR)-luciferase and LTR-green fluorescent protein cassette. The viral protein Tat was expressed after infection and integration of the HIV provirus into the genome of the host cell. HIV-1 transcription and expression of luciferase (CEM-M7) from the LTR promoter (viral promoter) were activated by the viral Tat protein. Therefore, luciferase activities determined three days post infection were directly proportional to the infection rates of the virus.

The experiments were carried out with HIV-1 NL4-3 wt virus and HIV-1 R263K virus which carries a mutation in the integrase gene rendering it resistant against dolutegravir. HIV-1 NL4-3 and HIV-1 R263K virus stock were generated by transient transfection of 293T cells with proviral DNA. Virus was harvested 2 days later, aliquoted and frozen at −80° C. The infectivity of virus stocks was determined by infecting CEM-M7 cells with serial virus dilutions and determining the luciferase activities 3 days later.

The antiviral compounds were stored and freshly dissolved before each experiment. To determine the antiviral activity of the compounds or combinations thereof, a total number of 1.5×10⁵ CEM-M7 cells were seeded in 150 μl medium in V-shaped microtiter plates. The respective dilutions of the antiviral compounds were prepared in PBS (7 dilutions and controls). Then, 10 μl of each compound or buffer controls were added to the cells. After an incubation time of 30 minutes, cells were infected with HIV-1 NL4-3 wt and HIV-1 R263K variant in a final volume of 200 μl.

Luciferase activities are shown as relative light units per second (RLU/s) which were derived from triplicate infections minus background activities derived from uninfected cells. The luciferase assay was performed as mentioned by the manufacturer (Luciferase Assay System, Promega, E1501) and quantified using an Orion Microplate Luminometer (Berthold). Raw data were exported to Excel and % infection rates were calculated. Graphs were created using GraphPad Prism. IC50 values were calculated by GraphPad Prism. The calculation of additive and synergistic effects was performed with the software computer language R as mentioned in the description from S. Loewe (Loewe 1953) by creating isobolograms. This analysis was performed in collaboration with the Applied Bioinformatics Group from Ulm University (PD Dr. Hans Kestler, Johann Kraus).

The following compounds were tested at the indicated mass ratios:

### REFERENCES

1. Combination known as Trii-572:
Dolutegravir/lamivudine/abacavir (50 mg/300 mg/600 mg)

### 2. Combination:

Dolutegravir/emtricitabine/tenofovir disoproxil fumarate (50 mg/200 mg/300 mg)
Combination according to the present invention:
Dolutegravir/emtricitabine/tenofovir disoproxil fumarate (50 mg/100 mg/150 mg)

### Results

The antiviral activity of dolutegravir in combination with two other antiviral drugs against HIV-1 NL4-3 wt and HIV-1 R263K was analyzed in CEM-M7 cells as mentioned above. An experiment with fixed drug combinations was performed. The drug concentrations causing 50% of the inhibition of the viral infection (IC50) were calculated using GraphPad Prism program and shown in Tables 1 and 2.

TABLE 1
Effect of different combinations/concentrations on WT infection

| Test Drugs | Concentrations [mg] | IC50 [nm] |
|---|---|---|
| DLT/LMV/ACV | 50/300/600 | 0.8 |
| DLT/EMT/TFV | 50/200/300 | 0.4 |
| DLT/EMT/TFV | 50/100/150 | 0.3 |

DLT corresponds to dolutegravir
LMV corresponds to lamivudine
ACV corresponds to abacavir
EMT corresponds to emtricitabine
TFV corresponds to tenofovir disoproxil fumarate

TABLE 2
Effects of different combinations/concentrations
on R263K infection

| Test Drugs | Concentrations [mg] | IC50 [nm] |
|---|---|---|
| DLT/LMV/ACV | 50/300/600 | 2.3 |
| DLT/EMT/TFV | 50/200/300 | 0.4 |
| DLT/EMT/TFV | 50/100/150 | 0.3 |

As can be seen from the above Tables 1 and 2, despite small amounts of emtricitabine and tenofovir disoproxil fumarate the triple combination according to the present invention shows an advantageous anti-viral activity (the lower the IC50 value the better the effect) against HIV-1 NL4-3 WT and against HIV-1 R263K being resistant against dolutegravir. Further, the combination shows unexpected and significant better anti-viral activity than the combination of the product Trii-572, which is promising in clinical trials and expected to be launched in the future.

It is expected that side effects can be reduce while maintaining the desired pharmacological effect.

1. Pharmaceutical combination comprising (a) dolutegravir, (b) emtricitabine and (c) tenofovir for use as a medicament, wherein the weight ratio of (a) dolutegravir and (b) emtricitabine is from 1:1 to 1:3.

2. Pharmaceutical combination for use according to claim 1, wherein the combination is used in the treatment of viral diseases, preferably in the treatment of an HIV-infection.

3. Pharmaceutical combination for use according to claim 1 or 2, wherein dolutegravir is used in the form of the sodium salt.

4. Pharmaceutical combination for use according to any one of the preceding claims, wherein emtricitabine is used in free form.

5. Pharmaceutical combination for use according to any one of the preceding claims, wherein tenofovir is used in form of a prodrug of tenofovir.

6. Pharmaceutical combination for use according to claim 5, wherein the prodrug of tenofovir is tenofovir disoproxil, tenofovir disoproxil fumarate, tenofovir alafenamide or hexadecyloxypropyl tenofovir.

7. Pharmaceutical combination for use according to claim 6, wherein the weight ratio of (a) dolutegravir and tenofovir disoproxil fumarate is from 1:2 to 1:5.

8. Pharmaceutical combination for use according to any one of the preceding claims, wherein the amount of dolutegravir administered is 20 to 75 mg per day.

9. Pharmaceutical combination for use according to any one of the preceding claims, wherein the amount of emtricitabine administered is 50 to 150 mg per day.

10. Pharmaceutical combination for use according to any one of the preceding claims, wherein the amount of tenofovir administered is 10 to 400 mg per day.

11. Pharmaceutical combination for use according to any one of the preceding claims, wherein the amount of tenofovir diisoproxil fumarate administered is 100 to 250 mg per day.

12. Pharmaceutical combination for use according to any one of the preceding claims, wherein the combination is administered once daily.

13. Pharmaceutical composition comprising a pharmaceutical combination according to any one of claims 1 to 11, wherein the composition comprises
(i) 20 to 75 mg dolutegravir,
(ii) 50 to 150 mg emtricitabine,
(iii) 0.5 to 400 mg tenofovir or a prodrug thereof and optionally
(iv) pharmaceutical excipient(s).

14. Pharmaceutical composition according to claim 13, wherein the composition is a solid oral dosage form, preferably a tablet.

15. A kit for the treatment of a viral infection, in particular of an HIV-infection, comprising
(i) 10 to 75 mg dolutegravir,
(ii) 50 to 150 mg emtricitabine,
(iii) 0.5 to 400 mg tenofovir or a prodrug thereof.
